(19) United States
(12) Plant Patent Application Publication
(10) Pub. No.: US 2004/0139517 P1
(43) Pub. Date: Jul. 15, 2004

(54) SANVITALIA SPECIOSA PLANT NAMED 'SANTASOL' (S&G 30038)

(76) Inventor: Anna M.W.P. Houbraken, Enkhuizen (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/342,730

(22) Filed: Jan. 15, 2003

(51) Int. Cl. A01H 5/00

(52) U.S. Cl. PLT/263

(57) ABSTRACT

A distinct cultivar of Sanvitalia plant named ‘Santasol’ (S&G 30038) which is particularly distinguished by its very dark foliage, compact plant habit, and constant floridity during summer, also under South European conditions.

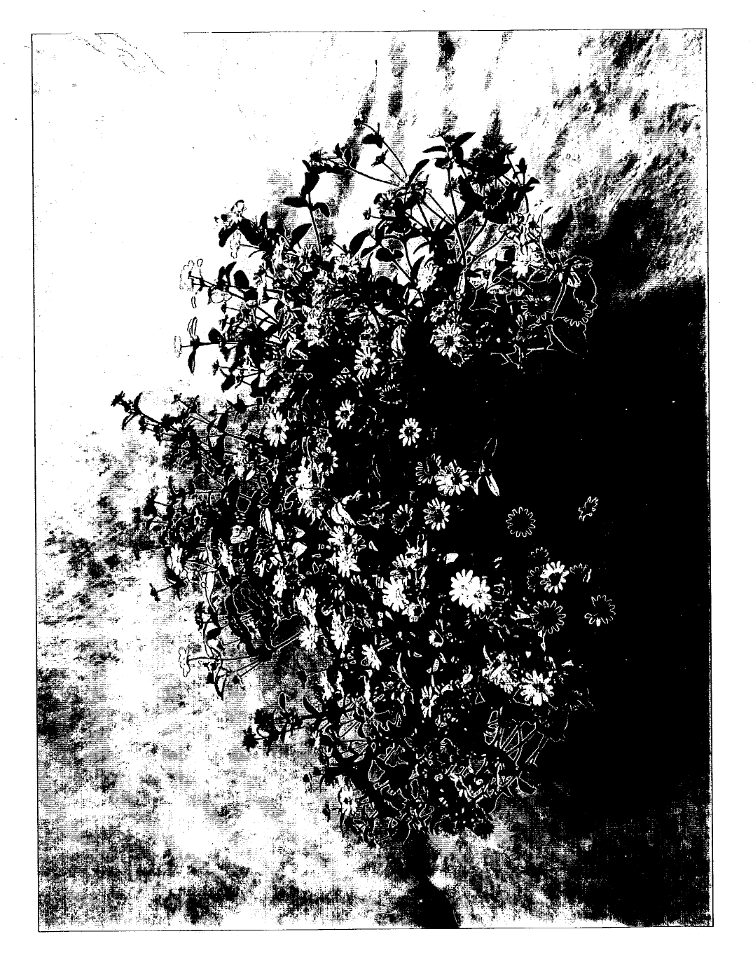

### VARIETAL DENOMINATION

‘Santasol’ (S&G 30038)

### BACKGRUND OF THE NEW PLANT

The present invention comprises a new distinct cultivar of Sanvitalia, botanically known as Sanvitalia×hybrida, native to South USA and Mexico. The new cultivar is propagated by cuttings resulting from a seedling from female plant Sanvitalia×hybrida identified as number ‘A 178.’ The male parent is unidentified as the seeding was a result of a non-controlled pollination.

### DESCRIPTION OF THE DRAWING

This new Sanvitalia plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Sanvitalia plant. The data which defines these characteristics were collected from asexual reproductions carried out in Enkhuizen, Netherlands. The plant history was taken on 27 weeks old plants, grown in an open field in Enkhuizen, Netherlands with day and night temperatures ranges from 15 to 30 degrees C., and night temperatures ranging from 5 to 18 degrees C.

Asexual reproduction of the new cultivar taken in a controlled environment in Enkhuizen, Netherlands since Septemeber 1999 has shown that the unique features of this new Sanvitalia are stable and reproduced true to type in successive generations.

Color references are primarily to the RHS Colour Chart of The Royal Horticultural Society of London.

Comparison between the new cultivar ‘Santasol,’ its
parent and a similar cultivar

| | ‘Santasol’ | Yellow Beauty | ‘Santis 99-3’ |
|---|---|---|---|
| Flower size | 1.3 | 1.6 | 1.6 |
| Internode length | 2.4 cm | 3.1 cm | 3.5 cm |
| Leaf color | 137B | 137A | 139A |

The plant:

Classification.—Botanical: Sanvitalia×hybrida cv. ‘Santasol’.

Parentage:

Female parent.—Seedling selection of Sanvitalia×hybrida, identified under number ‘A 178,’ which is not patented.

Male parent.—Unidentified section of Sanvitalia×hybrida.

Propagation:

Type of cuttings.—Terminal cuttings.

Time to initiate roots.—14 days at air temperature of 21 degrees C.

Time to develop roots.—21 days at air temperature of 17 degrees C.

Plant description:

Growth habit.—Spreading and trailing.

Plant height.—20-25 cm.

Vigour.—Vigorous.

Speading area of plant.—50 cm.

Strength.—Good tolerance to heat and cold conditions and diseases and pests.

Branching character.—Freely branching and lateral branching at every node after pinching.

Quantity of branches.—20 branches per plant.

Crop time.—11 weeks to flowering from good developed rooted cutting.

The stem:

Diameter.—2-3 mm.

Shape.—Cylindrical.

Anthocyan pigmentation.—Present.

Length of internode.—30-40 mm depending on light conditions.

Pubescence.—Present, shorter than 1 mm.

Color.—Young stem: N 77A — Older stem: N 77A.

The foliage:

Phyllotaxis.—Opposite.

Shape of leaf.—Lanceolate.

Leaf apex.—Rounded.

Leaf base.—Rounded.

Texture.—Sturdy hairy leaves.

Leaf length.—2.0-2.7 cm.

Leaf width.—0.7-1.2 cm.

Depth of incision.—No incision.

Color.—Upper side: 137B with red edge — Lower side 137C.

Pubescence.—Hirsute orientation with white hairs.

Petiole.—Length: No.

Venation.—Shape: Pinnate — Color: 137B

The flower bud:

Peduncle length.—2.0-2.5 cm.

Size of the bud.—Length: 0.2-0.3 cm — Diameter: 0.3-0.5 cm.

Shape.—Ball shape.

Color.—144B.

Inflorescence:

Diameter.—1.0-1.5 cm.

Form.—Capitulum with 1 series of ray florets and more series of disc florets.

Overlapping of ray florets.—No overlapping.

Receptacle.—Shape: Convex — Height: 2 mm — Color: 144A.

Involucre.—5 bracts in 1 series.

Color.—144A.

Fragrance.—Absent.

Number of flowers per plant.—400-500 flowers per plant.

Lastingness of flowers.—±25 days.

Ray florets:

Number of ray florets per inflor.—13.

Size.—Length: 4 mm — Width: 2 mm.

Shape of florets.—Ovate.

Color.—Between 9A and 14A.

Disc florets:

No. of disc florets per inflor.—>100.

Size.—Length: 2 mm — Width 1 mm.

Shape of florets.—Tubular.

Color.—153B.

Androecium:

Anther color.—153B.

Pollen color.—4A.

Amount of pollen.—Low.

Gynoecium:

Pistil number.—1 in each floret.

Style color.—9A.

Stigma color.—9A.

Seed:

Production.—10-13 seeds per capitulum.

Size.—Length: 2 mm — Width: 1 mm.

Color.—197A.

Physiological and ecological characteristics: Good tolerance to heat. Strong resistance to pests and diseases.

What is claimed is:

1. A new and distinct variety of Sanvitalia plant, substantially as illustrated and described herein.
